(19) United States
(12) Patent Application Publication
(10) Pub. No.: US 2015/0173847 A1
(43) Pub. Date: Jun. 25, 2015

(54) IMAGING MARKER AND UTILIZATION THEREOF

(71) Applicant: RIKEN, Saitama (JP)

(72) Inventors: Takuya Hayashi, Hyogo (JP); Iwao Nakajima, Tokyo (JP)

(21) Appl. No.: 14/399,440

(22) PCT Filed: Apr. 26, 2013
(86) PCT No.: PCT/JP2013/062499

§ 371 Date: Mar. 5, 2015

(30) Foreign Application Priority Data
May 8, 2012 (JP) 2012-107136

(51) Int. Cl. A61B 19/00; A61B 6/03; A61K 49/00; A61K 49/06; A61K 49/04; A61B 5/055; A61B 5/00

(52) U.S. Cl. CPC A61B 19/54; A61B 5/055; A61B 6/032; A61B 6/037; A61B 5/742; A61K 49/06; A61K 49/04; A61K 49/0002; A61B 2019/5433; A61B 2019/5495; A61B 2019/5454; A61B 2019/5466

(57) ABSTRACT

As an imaging marker for generating suitable contrasts in images taken through a plurality of medical diagnostic imaging techniques, a liquid containing a transition metal belonging to any of the fifth to seventh periods of the periodic table or a compound thereof in high concentration and/or high density or a composition containing the liquid is provided. Use of the composition easily generates a suitable contrast in any of the plurality of medical diagnostic imaging techniques.

### TECHNICAL FIELD

The present invention relates to an imaging marker that generates an appropriate contrast in a desired imaging technique and use of the imaging marker.

### BACKGROUND ART

Magnetic resonant imaging (hereinafter referred to also as “MRI”), positron emission tomography (hereinafter referred to also as “PET”), and computerized tomography (hereinafter referred to also as “CT”) are techniques that provide inside information on a subject (i.e. information on the inside of the body of the subject) as image information (both two-dimensional and three-dimensional). These techniques are widely used in the field of molecular imaging science as well as in the field of medicine. MRI allows image contrasts to be generated according to diverse in vivo pathological conditions by using various magnetic imaging or radio wave imaging procedures, and PET allows image contrasts to be generated according to diverse in vivo pathological conditions by administering, to a subject, various diagnostic agents labeled with radioisotopes and imaging an in vivo radioactivity distribution based on these diagnostic agents. CT irradiates the subject with X rays from outside and generates image contrasts according to the variation in X-ray absorption distribution through the subject. These image contrasts make it possible to medically diagnose the properties and pathological conditions of specific body tissues and scientifically detect changes in tissue characterization.

PET makes it possible to detect with high sensitivity a body distribution of agents labeled with radioisotopes (referred to also as “radioisotope-labeled agents”). However, due to the characteristics of an imaging apparatus, PET can only give images of comparatively low spatial resolution, and due to the distribution properties of agents, PET cannot give much anatomical information. Due to these disadvantages, PET has difficulty in accurate position identification. As compared with PET, MRI provides anatomical images of high spatial resolution, and is also high in accuracy of positional information. A combination of PET and MRI images makes it possible to multilaterally grasp pathological conditions and tissue characteristics in exact positions, and, as a result, enables highly accurate diagnosis.

However, these imaging techniques differ greatly from one another in terms of sensitivity, image contrast, resolution, positional information, and diagnostic usefulness. For example, in the case of MRI, an alteration of a condition for an imaging procedure brings about a change in image contrast. Further, in the case of PET, an image having a contrast only at a specific site can be generated by using a diagnostic agent that has a high specificity for a pathological lesion or a cell. For this reason, in the case of imaging the same subject, finding which site in the body of the subject has been imaged requires that a structure supposed to serve as a reference be recognized on both PET and MRI images, and the position of the target site is identified on the basis of a relative positional relationship with the reference structure.

Normally, the contours of the body surface of the subject are often seen (mostly due to non-specific integration). The same site in the internal structure is identified by relative positions with reference to the contours, and differences in contrast are recognized on the basis of the site thus identified. This as a result makes it possible to infer changes in pathological condition and changes in cell characteristic. However, a higher specificity of a PET diagnostic agent for a specific organ makes the body contours of the subject unclear, thus making it difficult to identify relative positions/shapes/contours inside the subject between images. Further, on the basis of the characteristics of imaging principles, different imaging conditions result in changes in distortion of images per se (especially in the case of MRI images) even with the object remaining exactly the same; therefore, there often occurs misregistration between images of different modalities. In this case, too, a structure supposed to serve as a reference needs to be recognized on the images of different modalities, and on that basis, position identification is made possible by a relative positional relationship with the target site.

For these reasons, correcting misregistration and/or distortion of the object that occurred between images obtained by different apparatuses and/or imaging techniques (registration) requires that a structure supposed to serve as a reference be recognized on images of different modalities.

As methods of position correction that involve combinations of images of different modalities, software methods and hardware methods are known. Known examples of software methods include a rigid-body site conversion method based on the homogeneity of a ratio of pixel values and/or the amount of mutual information (see Patent Literatures 1 to 3 etc. and Non-patent Literatures 1 and 2). This method can be simply used as a method of position correction between images of different contrasts for mathematically making position corrections by using only actual image information, and as such, is mostly widely used. However, this method is insufficient in position correction accuracy in a case where there is an extreme difference in contrast between images or in a case where there is much image noise. Known examples of hardware methods include: a method of identifying and correcting a motion of a subject by using an imaging apparatus to image the subject and at the same time image a marker attached/pasted to the subject and by using the marker as a structure to serve as a reference (see Patent Literatures 4 to 5, Non-patent Literatures 3 and 4, etc.); and a method of identifying and correcting a motion by using an infrared camera dedicated to monitoring motions (see Non-patent Literature 5). However, these techniques make the preparation etc. of apparatuses cumbersome. Further, there are also known techniques in which the shapes of markers per se (see Non-patent Literatures 6 to 8 etc.) are devised. However, these methods have had difficulty in selecting or preparing materials for optimum markers of high image contrast.

### CITATION LIST

### Patent Literature 1

Japanese Patent Application Publication, Tokukai, No. 2007-029502 A (Publication Date: Feb. 8, 2007)

### Patent Literature 2

Japanese Patent Application Publication, Tokukai, No. 2007-283108 A (Publication Date: Nov. 1, 2007)

### Patent Literature 3

Japanese Patent Application Publication, Tokukai, No. 2011-224194 A (Publication Date: Nov. 10, 2011)

### Patent Literature 4

Japanese Patent Application Publication, Tokukai, No. 2004-024582 A (Publication Date: Jan. 29, 2004)

### Patent Literature 5

Japanese Patent Application Publication, Tokukai, No. 2007-236837 A (Publication Date: Sep. 20, 2007)

### Patent Literature 6

U.S. Pat. No. 5,368,030 (Publication Date: Nov. 29, 1994)

### Patent Literature 7

United States Patent Application Publication No. 2011/105896 (Publication Date: May 5, 2011)

### Patent Literature 8

United States Patent Application Publication No. 2004/075048 (Publication Date: Apr. 22, 2004)

### Patent Literature 9

United States Patent Application Publication No. 2007/073143 (Publication Date: May 29, 2007)

### Non-Patent Literature 1

J Comput Assist Tomogr., 17(4), 536-546 (1993)

### Non-Patent Literature 2

IEEE Trans Med Imaging, 16(2), 187-198 (1997)

### Non-patent Literature 3

European Journal of Nuclear Medicine and Molecular Imaging, Vol. 30, No. 6, pp. 812-818 (2003)

### Non-patent Literature 4

Nuclear Medicine Communications, Vol. 28, No. 10, pp. 804-812 (2007)

### Non-patent Literature 5

IEEE Trans Nucl Science, 49(1), 116-123 (2002)

### SUMMARY OF INVENTION

### Technical Problem

Conventionally, the simplest and mainstream examples of methods of position correction are those based on software. However, position correction based on software is extremely low in accuracy in a case where there is an extreme difference in contrast between images of different modalities, in a case where there is much image noise, or in a case where the body contours of the subject are unclear as in the case of PET images generated by using a highly-specific diagnostic agent. In a method of position correction based on hardware, it is so complex and difficult to prepare and/or use a marker with a radiolabeling nuclide (i.e. an RI) present therein and/or equipment such as a camera dedicated to monitoring operation, and what is more, no electronic devices are not allowed in an MRI room, i.e. a high-magnetic-field environment. Therefore, such a hardware-based method has not been widely used. The markers disclosed in Patent Literatures 5 to 8 and Non-patent Literatures 3 and 4 make it necessary to produce markers with RIs present therein and has a possibility of errors being caused in image artifact and pixel value depending on the characteristics of imaging apparatuses (e.g. detection sensitivity, techniques used to reconstruct images, etc.) and the degree of integration of RIs in subjects, thus making it very difficult to prepare appropriate and minute concentrations of RIs. These conventional techniques are poor in practicality and reliability for identification of position with high accuracy between images. This has been one of the problems that are to be solved in undertaking medical diagnoses and pathological studies by multimodal imaging.

It is an object of the present invention to provide an imaging marker that is capable of easily generating a suitable image contrast for any of a plurality of imaging techniques (MRI, PET, and CT) without an RI present therein.

### Solution to Problem

In order to solve the foregoing problems, an imaging marker of the present invention includes a liquid containing a transition metal belonging to any of the fifth to seventh periods of the periodic table (excluding gadolinium) or a compound thereof. This makes it possible to generate a suitable image contrast for any of various imaging techniques. The imaging marker of the present invention may be formed with a container containing the liquid. Further, the imaging marker of the present invention may be used for registration of different images or as a phantom for position calibration of an imaging apparatus.

In the imaging marker of the present invention, it is preferable that the transition metal or the compound thereof be contained in high concentration in the liquid, and it is preferable that the transition metal or the compound thereof have a concentration of 100 mM or higher in the liquid. This makes it possible to generate a satisfactory image contrast in MRI. It is preferable the imaging marker of the present invention have a high density as a transition-metal compound solution, and it is preferable that the imaging marker of the present invention have a density of 1.2 g/mL or higher as a transition-metal compound solution. This makes it possible to generate a satisfactory image contrast in PET or CT. Further, in the imaging marker of the present invention, it is preferable that the transition metal solution have a T1 relaxivity of 0.1 mM⁻¹·sec⁻¹ or lower. This allows the imaging marker of the present invention to generate a satisfactory image contrast with not only part of an MRI image other than a subject but also part of the subject, thus making it possible to easily identify the position of the imaging marker.

In order to give data for use in diagnostic imaging, a method of the present invention includes the step of: (a) imaging a subject and an imaging marker with use of an imaging technique; and (b) generating an image of the subject and an image of the imaging marker, the imaging marker including a liquid containing a transition metal belonging to any of the fifth to seventh periods of the periodic table (excluding gadolinium) or a compound thereof.

In the method of the present invention, it is preferable that: in order to give plural items of data for use in diagnostic imaging on the basis of different imaging techniques, step (a) be executed a plurality of times with use of a different imaging technique every time; and step (b) be executed a plurality of times in correspondence with step (a). This makes it possible to obtain MRI images and/or CT images and PET images through a series of processes.

It is preferable that the method of the present invention further include the step of (e) superimposing a plurality of images of the imaging marker that were generated by executing step (a) a plurality of times. This enables the accurate registration of MRI images and/or CT images and PET images.

A system of the present invention includes: an imaging marker including a liquid containing a transition metal belonging to any of the fifth to seventh periods of the periodic table (excluding gadolinium) or a compound thereof; a holding section that holds a subject to be imaged; an imaging section that images the subject and the imaging marker; an image generation section that generates an image of the subject and the imaging marker; and a display section that displays the image of the subject and the image of the imaging marker as a single image.

In the system of the present invention, the imaging section may comprise a plurality of imaging sections, in which case the plurality of imaging sections correspond to different imaging techniques, respectively. Further, under different imaging conditions (e.g. TR values, TE values, sequence types, etc. in MRI, radiolabeled diagnostic agents etc. in PET), even imaging with the same imaging section corresponds to different imaging techniques. Further, it is preferable that the display section display, as a single image, an image of the subject and an image of the imaging marker that were formed through the same imaging technique, and it is more preferable that the display section register, superimpose, and display images of the imaging marker that are contained in a plurality of single images corresponding to the different imaging techniques.

### Advantageous Effects of Invention

The accuracy and precision of registration can be increased by identifying the imaging marker of the present invention on images taken through imaging techniques of different modalities. When used in registration, the marker needs only be imaged through a desired imaging technique after being pasted to the body surface of the subject or to an area around the subject; therefore, this registration method is much simpler technically than the method involving the use of a conventional RI maker and the registration method involving the use of an infrared camera, and is superior to the software-based registration methods in terms of always enabling reliable registration. This makes it possible to view a plurality of images in exact positions, thus enabling the accurate diagnosis of diseases and the accurate grasp of pathological conditions. Therefore, the imaging marker of the present invention is useful for radiological diagnosis and imaging study. Further, the imaging marker of the present invention enables the identification of pathological conditions in exact positions, and as such, is useful in therapeutic applications such as irradiation under image information guidance. Further, use of such an imaging marker of the present invention makes it possible to simplify the verification of image position accuracy and distortion of multimodal imaging apparatuses, the calibration of multimodal imaging apparatuses, and the development of multimodal imaging apparatuses.

Further, the imaging marker of the present invention can be used as an in vivo marker for position identification by being injected into a specific site in the body or administered into a bleed vessel, and is also usable as an MRI contrast medium and as a PET diagnostic agent (imaging probe). The imaging marker of the present invention makes it easy to obtain accurate position-corrected images, and also makes it possible to achieve the accurate understanding of biological phenomena and the accurate diagnosis of various diseases.

Furthermore, the imaging marker of the present invention is usable as a material for a phantom. Since high-contrast images of the same shape are generated by using the same phantom, the imaging marker of the present invention can be used for imaging aimed at the calibration of position accuracy and precision of MRI and PET and/or CT and the correction of misregistration of MRI and PET and/or CT.

### BRIEF DESCRIPTION OF DRAWINGS

FIG. 1 is a diagram showing the association between the concentrations of metals in aqueous solutions and image contrasts.

FIG. 2 is a diagram showing the association between the densities of the aqueous solutions used in FIG. 1 and image contrasts.

FIG. 3 is a diagram showing results of multimodal imaging (MRI, PET, and CT) performed on various markers containing various transition-metal compound aqueous solutions.

FIG. 4 is a diagram showing results of position correction performed on images taken through imaging techniques of different modalities (PET and MRI) with multimodal imaging markers attached to areas around animals.

### DESCRIPTION OF EMBODIMENTS

In magnetic resonance imaging (MRI), a substance placed in a high-magnetic-field is irradiated with radio waves at specific frequencies, and the radio waves resonate with the gyrating movements of protons (hydrogen atoms) in the substance to cause the protons to be shifted into a high-energy state, then returned to a low-energy state, the process called as relaxation, by emitting radio waves. By detecting the radio waves that are emitted in this relaxation process, images can be obtained. Since the molecules of water in a living organism contain an abundance of proton atoms, the relaxation phenomenon of protons varies depending on the chemical state of the water molecules, and this variation effects contrast of image. For example, the relaxation time of protons is very long in a case where distilled water, which takes the form of a liquid at room temperature, is imaged in a container, but in a case where an an aqueous solution containing a magnetic substance (e.g. a paramagnetic substance) is present in the vicinity of the protons, the relaxation time of protons shorten, whereby an image contrast is generated. Relaxation has two independent physical characteristics, namely longitudinal relaxation (T1 relaxation) and transverse relaxation (T2 relaxation). Image contrasts of MRI images are roughly classified into T1-weighted images (i.e. images in which differences in T1 relaxation are weighted) and T2-weighted images (i.e. images in which differences in T2 relaxation are weighted). Among various MRI scanning techniques, 3D-T1-weighted imaging techniques (such as FSPGR and MPRAGE) based on the gradient echo technique are often used as imaging procedures for MRI that provide much anatomical information and that enable high-speed and high-resolution imaging.

A transition metal is one of those substances which, due to their tendency to have stable unpaired electrons, easily exhibit magnetic properties such as paramagnetism and ferromagnetism. Paramagnetism is a property exhibited by substances which are not magnetized in the absence of an external magnetic field but, when placed in an environment in which a magnetic field is present, are magnetized parallel to the field. The presence of a transition-metal element in the vicinity of water molecules shortens the relaxation time of the water molecules (as compared with the case of free water alone), whereby contrasts are generated in MRI images, especially T1-weighted images.

Therefore, in general, aqueous solutions of compounds of transition metals such as nickel, copper, iron, manganese, and gadolinium are often used as materials for MRI phantoms and contrast media. When aqueous solutions of these transition-metal compounds are used in MRI, image contrasts on T1-weighted images can be generated by using the aqueous solutions under comparatively low concentration conditions (10 mM or lower), as the transition-metal compounds have the properties of being high in relaxivity (i.e. the ability to generate a contrast in MRI per unit concentration). However, higher concentrations (100 mM or higher) of these transition metals lead to extremely weaker signals. This is because T2 relaxation is also extremely accelerated under such conditions. For example, as will be mentioned in the Examples below, an abrupt decrease in MRI signal value is observed when a concentration of nickel in an aqueous solution exceeds 5 to 10 mM, and an abrupt decrease in MRI signal value is observed when a concentration of gadolinium in an aqueous solution exceeds 1 to 5 mM. Such concentrations are much lower than the saturating concentration of any of the metals. As just described, the transition metals conventionally used in MRI suitably generate contrasts in MRI images by being used in low concentration.

The inventors of the present invention accomplished the present invention by finding the presence of transition metals which, when used at conventional concentrations of 10 mM or lower, do not generate contrasts in MRI images but, only when used at very high concentrations of 100 mM of higher, generate suitable image contrasts. The present invention could not have easily been found by a person skilled in the art on the basis of the aforementioned general common technical knowledge.

An embodiment of the present invention is described below. It should be noted the present invention is not limited to this embodiment.

(1. Imaging Marker)

The present invention provides an imaging maker that is suitable to generate a contrast in an MRI image. An imaging marker of the present invention is in such a form that a transition-metal element is present in the vicinity of water molecules. Specifically, the imaging marker of the present invention is in the form of a liquid containing a transition metal or a compound thereof, or may be formed with a container containing the liquid. As used herein, “a transition metal or a compound thereof” is intended to mean “a metal or a metal compound” that provides a transition metal that should be present in the vicinity of the molecules of water in the imaging marker of the present invention in liquid form, or may be a simple transition metal or a salt of a transition metal.

In the imaging marker of the present invention, a transition metal that is suitably present in the vicinity of the molecules of water in the liquid needs only be a transition metal belonging to any of the fifth to seventh periods of the periodic table, and is preferably a transition metal belonging to the sixth period of the periodic table (excluding gadolinium). Since conditions under which a transition-metal element generates a contrast in MRI depend on a large number of factors such as the magnetic properties and chemical state of a metal compound of the element, the compound concentration, the temperature, the magnetostatic field strength of MRI, and the imaging technique and procedure, a person skilled in the art cannot easily conceive of the idea that a transition metal or a compound thereof that is to be contained in the present invention successfully generates a contrast in an MRI image. Further, the transition metals conventionally used in MRI, when used in higher concentration in solutions, become unable to give satisfactory image contrasts. Therefore, no attempt has been made to use any of those transition metals in high concentration for MRI. The transition metal that is to be contained in the present invention generates a satisfactory MRI signal by being used at a high concentration that is much higher than the range of concentrations of the general transition metals named above. This matter, too, cannot be easily conceived of by a person skilled in the art. For these reasons, the imaging marker of the present invention cannot be easily conceived of by a person skilled in the art.

Thus, the imaging marker of the present invention is in the form of a liquid containing a high concentration of a transition metal or a compound thereof. The concentration, in the liquid, of the transition metal or the compound thereof that is contained in the imaging marker of the present invention is preferably 100 mM or higher, more preferably 500 mM or higher, even more preferably 1000 mM or higher, or may be a saturating concentration.

In PET and CT, image contrasts are generated by detecting gamma rays emitted from the body or X-rays exogenously radiated. The transmittance of gamma rays (including X-rays) varies depending of the properties of a substance by which they are to be transmitted. For example, the absorptance of gamma rays in a single substance depends on the frequency of the gamma rays. In general, however, the higher the density of a substance is, the higher the absorptance is. In a case where the absorbance of gamma rays (or X-rays) is high, satisfactory contrasts are generated in both PET transmission images and CT images. Since the imaging marker of the present invention contains a high concentration of a metal that is great in atomic weight or a compound thereof, such as a transition metal belonging to any of the fifth to seventh periods of the periodic table (especially a transition metal belonging to the sixth period of the periodic table), the imaging marker of the present invention has a very high density as a transition-metal compound solution. Thus, the imaging marker of the present invention is a multimodal imaging marker that not only generates a contrast in an MRI image but also generates satisfactory contrasts in both a PET image and a CT image. For example, tungsten, which was used in the Examples, is a heavy metal, atomic number 74, that is very high in solubility to water as compounds such as sodium polytungstate, and aqueous solutions of these compounds have a maximum density of 3.08 g/mL, are high in gamma-ray absorptive power, and are usable as marker materials that generate high contrasts in PET and/or CT.

The imaging marker of the present invention is in the form of a high-density liquid containing a transition metal or a compound thereof. The density of the imaging marker of the present invention in the form of a transition-metal compound solution contained therein is preferably 1.2 g/mL or higher, more preferably 1.3 g/mL or higher, even more preferably 1.4 g/mL or higher. As will be mentioned in the Examples below, a solution of any of those transition metals (such as nickel, copper, and gadolinium) commonly used as phantoms and contrast media in MRI has a density of about 1 g/mL no matter what concentration the solution may have, and an attempt to prepare a high-density solution of any of those transition metals is met with difficulty in obtaining a solution having a density of 1.25 g/mL or higher, as such an attempt ends up in the solution exceeding its saturating concentration. Such a solution, even if obtained at all, no longer generates any MRI signal.

It should be noted that use of high-density and high-concentration transition-metal compound solutions as PET and/or CT markers is unknown. Conventionally, solutions containing RIs have been used to collect PET emission images. However, since three is a possibility of errors being caused in image artifact and pixel value depending on the characteristics of imaging apparatuses (e.g. detection sensitivity, techniques used to reconstruct images, etc.) and the degree of integration of RIs in subjects, it is very difficult to prepare appropriate concentrations of RIs. Further, it is so complex and difficult to handle minute amounts of RIs. Even for a person skilled in the art who has conceived of usability of high-density transition-metal compound solutions as PET and/or CT markers, it is by no means easy to predict that image contrast can be generated in MRI as in the case of the imaging marker of the present invention.

Thus, the present invention can provide a fiducial marker for identifying the position of an object in a plurality of imaging techniques such as MRI, PET, and CT. Use of such a marker enables the accurate grasp of the position of the object, and is believed to be useful for imaging study, clinical diagnostic imaging, radiological diagnosis, etc. Imaging an object with such a marker material as a phantom is useful, for example, for calibration of positional distortion and/or accuracy attributed to the type of imaging apparatus and to differences in imaging condition or for verification of such positional distortion and/or accuracy.

A preferred form of a liquid in the present invention may be an aqueous solution, a dispersion liquid (such as a colloidal solution, a gel, or a sol), a suspension, an emulsion, or the like, and a preferred medium is water.

Since the imaging marker of the present invention is used in medical practice, it is preferable, for the sake of low-cost manufacturing, that the transition metal to be used be Hf, Ta, W, Re, Os, or Ir, and it is preferable, for the sake of safe use, that the transition metal to be used be Hf, Ta, W, Re, Os, Ir, Pt, or Au, more preferably W (tungsten), which are non-toxic or mildly toxic. Especially in the case of a compound of tungsten, as will be mentioned in the Example below, a higher concentration of the compound in a solution leads to a higher MRI signal value. Further, since the compound is very high in solubility to an aqueous solution, image contrasts are generated by gamma-ray absorption in PET and/or CT.

MRI, PET, and/or CT images are taken after attaching or pasting, to the body surface of a subject or to a retainer that supports the subject, a container made of resin or the like and tightly sealing in the imaging marker of the present invention. In the case of MRI, the effect of shortening of relaxation time allows the marker to be seen in the same image as the subject under most MRI imaging conditions. In the case of PET, the marker is seen on an image for use in gamma-ray absorption correction (transmission image). The term “transmission image” here means an image obtained by imaging preceding the administration of a PET-diagnostic radionuclide-labeled diagnostic agent to the subject, and an image that is obtained by imaging following the administration of the PET-diagnostic radionuclide-labeled diagnostic agent to the subject is referred to as an “emission image”. Measuring the distribution of gamma-ray absorption in the subject of imaging in advance makes it possible to make an absorption correction to the emission image and obtain highly quantitative/homogeneous images for the actual concentration distribution of the diagnostic agent. In the case of CT, too, the marker absorbs X-rays in a similar manner to the principle of PET and therefore exhibits an image contrast.

Since the same marker is seen with the subject on images of different modalities, it is possible to register the images of different modalities by identifying (barycentric) positions of the maker and shifting or rotating them of one image modality to the same site of the other. Forming the marker in a highly spatially symmetric shape (ideally in a spherical shape) in advance increases the accuracy with which the barycentric positions are identified, thus enabling identification with position accuracy higher than that would be attained with the actual size (e.g. a diameter of approximately 2 to 4 mm) of the marker and thus making it possible to perform position correction with accuracy equal to or higher than that would be attained with the size of the marker. Theoretically, a shift in position in a space is uniquely defined with at least three markers spatially present. In actuality, however, errors of measurement (such as distortion of images) and errors of position identification (i.e. errors of identification of barycentric positions of the markers) are passed onto errors of position correction. Therefore, the larger the number of markers is, the higher the actual position correction accuracy is. A degree of shift in position is calculated by calculating an optimum solution by the least squares-method of position correction of positional information (sequence of points) of a plurality of markers.

As used herein, the term “subject” is intended to mean a human and a non-human animal that serve as objects to be imaged, and the non-human animal is not limited to a mammal. The term “subject of imaging” is intended to mean an object to be imaged that is not limited to a living organism, and encompasses the term “subject”.

In a case where the imaging marker of the present invention is put into a container, the container to be used is not particularly limited, and may be one used for any of the publicly-known MRI markers. For use as a PET diagnostic agent, the imaging marker of the present invention may be tightly sealed in by a technique publicly known in this technical field. Examples include, but are not limited to, those forms shown in the Examples below (an extremely small cylindrical container to which a lid is welded or screwed after the marker has been placed in the container), capsules (e.g. Japanese Patent Application Publication, Tokukaihei, No. 5-31352 A, Japanese Patent Application Publication, Tokukaihei, No. 5-245366 A, Japanese Patent Application Publication, Tokukai, No. 2003-325638 A, etc.), a disk-shaped container (Patent Literature 6), and spherical and/or mortar-shaped containers (Patent Literatures 5 and 8).

As mentioned above, the liquid form of the imaging marker of the present invention may be a dispersion liquid (such as a colloidal solution, a gel, or a sol), a suspension, an emulsion, or the like, as well as an aqueous solution. Since the imaging marker of the present invention is prepared by mixing, with a medium, a metal compound containing any of the aforementioned transition metals, a high-density solution can be prepared by simply using a metal compound that is high in solubility to the target medium. Without being so limited to a combination of a specific metal compound and a specific medium, the imaging marker of the present invention needs only be a composition that is in the form of a high-density liquid containing any of the aforementioned transition metals or a compound thereof. In particular, the present invention as a multimodal imaging marker needs only be directed to a composition that is in the form of a high-concentration and high-density liquid containing any of the aforementioned transition metals or a compound thereof.

A high-density solution is high in specific gravity, and a high-density solution having a specific gravity of 2.2 or higher is used as a so-called heavy solution in divergent selection and specific gravity measurement of minerals. Known examples of such heavy solutions are iodomethane (CH₃I), tin tetrachloride (SnCl₄), dibromomethane (CH₂Br₂), manganese tetrafluoride (MnF₄), tin dichloride (SbCl₂), bromoform (CHBr₃), carbon tetrabromide (CBr₄), tetrabromoethane (Br₂CHCHBr₂), BaHgBr₄+H₂O, CH₂I₂, SnBr₄, CH₂I₂CHI₃, a mercury potassium iodide solution (K₂HgI₄+H₂O), WF₄, HCO₂Tl+H₂O, SnI₄, BaHgI₄+H₂O, AgTl(NO₂)₂*H₂O, thallium malonate formate (HCO₂Tl+C₂H₂O₄Tl₂+H₂O), HgTl(NO₃)₂+H₂O, an aqueous solution of sodium polytungstate (SPT), an aqueous solution of lithium heteropolytungstate (LST), lithium metatungstate (LMT), etc. Of these heavy solutions, transition-metal-containing heavy solutions are: (1) MnF₄ and WF₄; (2) AgTl(NO₃)₂+H₂O; and (3) tungsten solutions such as SPT solution, LMT solution, and LST solution. However, the heavy solutions (1) do not produce signals in MRI, as they do not contain protons, and the heavy solution (2) is not suitable to MRI imaging for humans or animals, as it is harmful. The tungsten compounds (3) are all known as heavy solutions that dissolve in high concentrations in water and that are highly safe for living organisms.

Among them, sodium polytungstate (SPT), also referred to also as “hexasodium tungstate” or “sodium metatungstate”, has molecular formula 3Na₂WO₄.9WO₃·H₂O, with molecular weight 2986.1 g/mol, and is high in water solubility (>1 g/mL) as in the Examples, and makes it possible to prepare an aqueous solution having a high density of at most 3.08 g/mL at 20 to 25° C., and is high in safety. The preparation of such a solution with SPT makes it possible to easily adjust the concentration of tungsten in the imaging marker of the present invention according to the image contrast intensity that a user of the present invention needs. Further, the LMT, too, is suitable to preparing a similarly high-density solution. As just described, a metal compound that is used to prepare the imaging marker of the present invention may be a publicly-known compound with which a heavy solution is prepared. The preparation of such an aqueous solution with SPT makes it possible to make an imaging marker appropriate to the image contrast intensity that a user of the present invention needs.

Further, LST (lithium heteropolytungstate), too, is suitable to preparing a similarly high-density aqueous solution, and as such, can be used as a material for the imaging marker of the present invention. Further, an aqueous solution of ammonium metatungstate, an aqueous solution of phosphotungstic acid, an aqueous solution of silicotungstic acid, an aqueous solution of phosphomolybdic acid, and an aqueous solution of ammonium molybdate, which are not commonly used as heavy solutions, make it possible to prepare an aqueous solution containing a transition metal such as tungsten or molybdenum and having a specific gravity of 1.2 or higher, and as such, can each be used as a material for the imaging marker of the present invention.

Further, besides tungsten, there are transition metals (compounds) that have low-relaxivity properties. Among them, examples of transition metals having comparatively low magnetic susceptibility as simple substances include molybdenum (Mo), osmium (Os), hafnium (Hf), rhenium (Re), tantalum (Ta), technetium (Tc), etc.

(2: Use of an Imaging Marker)

The present invention provides a method of obtaining data for use in diagnostic imaging. The method of the present invention needs only include the step of (a) imaging a subject and an imaging marker with use of an imaging technique, and for obtaining data usable in diagnostic imaging, it is preferable that the method of the present invention further include the step of (b) generating an image of the subject and an image of the imaging marker. The imaging marker for use in the method of the present invention needs only be a composition including a liquid containing a transition metal belonging to any of the fifth to seventh periods of the periodic table or a compound thereof, and it is preferable that the liquid have a high density, more preferable that the liquid contains the transition metal in the form of a high-density liquid, and even more preferable that the transition metal have a low relaxivity (especially a T1 relaxivity of 0.1 mM⁻¹·sec⁻¹ or lower).

During execution of the method of the present invention, it is preferable that both the subject and the imaging marker be present in the same imaging region. That is, it is preferable that during imaging, the imaging marker be attached or pasted to a body surface of the subject or to a holding member that holds the subject, and it is more preferable that the method of the present invention further include the step of (c), prior to step (a), attaching or pasting the imaging marker to a body surface of the subject or to a holding member that holds the subject. It should be noted that use of the present invention makes it possible to execute all of the steps without removing the imaging marker (first marker) thus attached or pasted.

As mentioned above, the imaging marker is also usable as a marker that is injected into the body for use, an MRI contrast medium, and as a PET diagnostic agent. For this reason, the imaging marker may be introduced as a second marker into the body of the subject during imaging, and the method of the present invention may further include the step of (d), prior to step (a), introducing or injecting the imaging marker into a body of the subject.

In a case where the imaging marker functions as a multimodal imaging marker, step (a) of the method of the present invention may be executed a plurality of times with use of a different imaging technique every time; and step (b) of the method of the present invention may be executed a plurality of times in correspondence with step (a). In this case, it is preferable that the method of the present invention further include the step of (e) superimposing a plurality of images of the imaging marker that were generated by executing step (a) a plurality of times. This enables the accurate registration of a plurality of images obtained through different imaging techniques.

The present invention may be targeted at any kinds of diseases without any particular limitations. PET is often used for diagnosis of cancer, and is also used for diagnosis of nervous and mental diseases (such as Alzheimer's disease, cerebral stroke, Parkinson's disease, and schizophrenia). Although PET has difficulty in grasping the position of a lesion, registration with MRI images and/or CT images with use of a multimodal imaging marker makes it possible to accurately identify the anatomical position of the lesion, and to more accurately determine the nature of and diagnose the involved site from the integration characteristics of a plurality of PET images. Furthermore, the present invention is usable for treatment as well as diagnosis. Making a treatment regimen for cancer radiotherapy with use of PET images and markers has already been proposed (e.g. see THE JOURNAL OF NUCLEAR MEDICINE Vol. 45, No. 7, p. 1146-1154 (2004)), and use of the present invention is extremely helpful in a surgical plan and/or in a regimen for radiotherapy such as a gamma knife on the basis of images of high spatial resolution. That is, the data “for use in diagnostic imaging” of the present invention is not limited to data for diagnosing the presence or absence of a disease or the degree of progression of a disease, but encompasses data for planning treatment for a disease.

Furthermore, the present invention provides a system for use in diagnostic imaging. The system “for use in diagnostic imaging” of the present invention is not limited to a system configured to diagnose the presence or absence of a disease or the degree of progression of a disease, but encompasses a system configured to plan treatment for a disease.

The system of the present invention includes a holding section, an imaging section, a storage section, an image generation section (CPU), and a display section as its functional blocks. The imaging section functions to image a subject (and an imaging marker) held by the holding section. The CPU functions as an arithmetic section to generate an image. The storage section functions to store information obtained by the imaging section to be processed by the arithmetic section. The display section functions to display an image generated by the arithmetic section. It should be noted that these functional blocks are realized by the CPU executing an image generation program stored in the storage section and controlling a peripheral circuit such as an input/output circuit (not illustrated). Further, during execution of the program, an imaging marker including a liquid containing a transition metal belonging to any of the fifth to seventh periods of the periodic table or a compound thereof is attached or pasted to a body surface of the subject or to the holding section, or is introduced into a body of the subject.

In the system of the present invention, the imaging section uses an imaging technique to image the subject held by the holding section. For generation of an image of the subject, information obtained by imaging is temporarily stored in the storage section. Upon completion of imaging, the CPU takes out the information stored in the storage section and generates an image of the subject. The image thus generated may be outputted directly to the display section, or may be temporarily stored in the storage section.

In a case where the imaging technique used is MRI or CT, the imaging section of the system of the present invention images an imaging marker placed in an imaging region (i.e. a first marker attached or pasted to the body surface of the subject or to the holding section), at the same time as it images the subject held by the holding section. That is, the aforementioned information contains not only information from the subject but also information from the first marker. Moreover, an image generated of the subject by the CPU contains an image of the first marker, too. Accordingly, the display section displays the outputted image of the subject as a single image containing an image of the subject and an image of the first marker. Further, both MRI imaging and CT imaging may be performed under any imaging conditions as long as the final output takes the form of an image.

In PET, information for generating an image for use in absorption correction (transmission image) is obtained before information for generating an image of a PET diagnostic agent (emission image) is obtained. In a case where the imaging technique used is PET, the display section displays, as an absorption image, a single image containing an image of the subject and an image of the first marker.

It should be noted that since the imaging marker of the present invention is also usable as a PET diagnostic agent, the imaging marker can be used as a second marker that is introduced into the body of the subject. In this case, regarding a subject into whose body the second marker has been introduced after imaging for a transmission image, the display section displays, as an emission image, a single image containing an image of the subject and an image of the first marker.

In a case where the imaging marker of the present invention is a multimodal imaging marker, the system of the present invention may include a plurality of imaging markers. In this case, one imaging section corresponds to MRI, and another imaging section corresponds to PET. Moreover, the CPU generates an MRI image of the subject on the basis of information obtained from the imaging section corresponding to MRI and outputs the MRI image to the display section, and generates a PET image of the subject on the basis of information obtained from the imaging section corresponding to PET and outputs the PET image to the display section. The MRI image of the subject contains an MRI image of the first marker, and the PET image of the subject contains a PET image of the first marker. Therefore, the display section displays the outputted MRI image of the subject as a single image containing the MRI image of the subject and the MRI image of the first marker, and displays the outputted PET image of the subject as a single image containing the PET image of the subject and the PET image of the first marker.

In the system of the present invention, the MRI and PET images of the subject may be superimposed. In this case, the CPU performs position correction on the MRI and PET images of the first marker, and the display section superimposes and displays the corrected MRI and PET images of the first marker. The position correction includes: identifying the barycenter of each marker on each of the images visually, manually, or by automatic processing based on software; and performing registration on the basis of these barycenters.

It should be noted that a system that is used for executing the aforementioned method of obtaining data for use in diagnostic imaging falls within the scope of the present invention. The embodiment is described by taking, as an example, a case where members constituting a system according to the present invention are “functional blocks that are realized by an arithmetic section such as a CPU executing a program code stored in a recording medium such as ROM or RAM”. However, the functional blocks may alternatively be realized by hardware that performs the same processes. Further, the functional blocks can alternatively be realized by a combination of hardware that performs some of the processes and the arithmetic section, which executes a program code for performing control of the hardware and the remaining processes. Furthermore, even those ones of the members where are described as hardware can be realized by a combination of hardware that performs some of the processes and the arithmetic section, which executes a program code for performing control of the hardware and the remaining processes. It should be noted that the arithmetic section may be a single arithmetic section, or a plurality of arithmetic section connected to each other via buses inside the apparatus and/or various communication paths may execute a program code in cooperation with each other.

The embodiments of the present invention are described in more detail with reference to the Examples below. Further, all of the documents cited herein are incorporated herein by reference.

### EXAMPLES

Manganese (Mn), nickel (Ni), copper (Cu), and gadolinium (Gd) are transition metals, and aqueous solutions of compounds thereof have been used as materials for MRI phantoms and raw materials for contrast media, as these aqueous solutions generate excellent image contrasts in MRI. However, it is unknown what kinds of images are generated by these aqueous solutions in imaging techniques, such as positron emission tomography (PET) and computerized tomography (CT), in which image contrasts are obtained by the gamma-ray or X-ray absorption differences. Therefore, aqueous solutions containing any of the metals (Mn, Ni, Cu, and Gd) in varying concentrations were examined for their image contrast properties in PET absorption images. Furthermore, aqueous solutions of tungsten, which is a transition metal belonging to the sixth period, of the Examples of the present invention were examined for image contrasts in MRI and PET, and were compared with the conventional products. Further, as for some of the aqueous solutions, CT images were taken for examination of image contrasts.

Manganese dichloride (MnCl₂; Wako Pure Chemical Industries, Ltd.) was used as a compound of manganese (Mn), and aqueous solutions of this compound in metal concentrations of 1000 mM, 100 mM, 10 mM, 1 mM, 0.1 mM, and 0.01 mM were prepared.

Copper sulfate (CuSO₄; Wako Pure Chemical Industries, Ltd.) was used as a compound of copper (Cu), and aqueous solutions of this compound in metal concentrations of 1000 mM, 100 mM, 10 mM, 1 mM, 0.1 mM, and 0.01 mM were prepared.

Nickel sulfate (NiSO₄; Wako Pure Chemical Industries, Ltd.) was used as a water-soluble compound of nickel (Ni). Aqueous solutions of the nickel compound in varying concentrations of 2000 mM, which is close to the saturating concentration, 1500 mM, 1000 mM, 500 mM, 100 mM, 50 mM, 10 mM, 5 mM, 1 mM, 0.5 mM, 0.1 mM, 0.05 mM, and 0.01 mM were prepared.

A medically-used aqueous solution of meglumine gadopentetate (Gd-DTPA; Bayer Yakuhin, Ltd.) was used in this example experiment as a gadolinium (Gd) containing compound. This aqueous solution was prepared in varying concentrations of 100 mM, 50 mM, 10 mM, 5 mM, 1 mM, 0.5 mM, 0.1 mM, 0.05 mM, and 0.01 mM. An undiluted solution of meglumine gadopentetate having a high concentration of 500 mM was also prepared.

Sodium polytungstate (SPT; Wako Pure Chemical Industries, Ltd.) and lithium heterotungstate (LST; Central Chemicals Consulting Pty Ltd., Australia) were used as water-soluble compounds of tungsten (W). Tungsten aqueous solutions of SPT in tungsten concentrations of 9357 mM, 8864 mM, 8372 mM, 6970 mM, 4924 mM, 2970 mM, 985 mM, and 98 mM were prepared. An almost-saturated tungsten aqueous solution of SPT in a concentration of 820 mM (estimated concentration; 9850 mM in tungsten concentration) was also prepared. Further, aqueous tungsten solutions of LST in tungsten concentrations of 8930 mM, 4465 mM, 893 mM, and 89 mM were also prepared.

After the aqueous solutions were put into each separate tube (2 mL), the tubes were hermetically sealed, placed on a fixed base made of styrene foam, and imaged through MRI and PET. Further, the weight of each of the solutions was calculated by measuring the weight of each of the tubes before and after the solution was put into the tube, and the density of the solution was calculated.

As controls, a tube sealing in 2 mL of pure water and a commercially available multimodal imaging marker (IZI medical products, MD, USA) were used.

For PET imaging, transmission images were acquired by using a 3D-PET apparatus (microPET; manufactured by Siemens AG) and a ⁶⁸Ga/Ge point source (30-minute imaging). The images were reconstructed by the back projection method, and an image of absorption coefficient values was computed. A region of interest was set in each of the tubes of solution in the image. The average of absorption coefficient values within the region of interest was obtained. A PET image contrast ratio (PET-CNR) based on reference to water was obtained by Formula (1):

[Math. 1]

PET-CNR=(“Average of Absorption Coefficient Values within ROI of Metal Aqueous Solution Part”−“Average of Absorption Coefficient Values within ROI of Pure Water Part”)/“Standard Deviation of Absorption Coefficient Value in ROI of Background Part” (1)

MRI imaging was performed by the Magnetization-Prepared Rapid Gradient-Echo (MPRAGE) technique using a 3-tesla MRI apparatus (Allegra; manufactured by Siemens AG). This imaging procedure is an imaging procedure that is frequently used in obtaining anatomical images, and can mainly give T1-weighted image contrasts. Imaging with the MPRAGE technique was performed under the following conditions: TR 1300 msec; TE 4.74 msec; inversion time (T1) 1030 msec; flip angle 8°; matrix 192×192; field of vision (FOV) 100 mm; and slice thickness 1.5 mm. Imaging was performed in a constant environment at a room temperature of 22° C. After the tubes of compound were placed and imaged, a region of interest was set in each of the tubes. The average of MRI signal values within the region of interest was obtained. An MRI image contrast ratio (MRI-CNR) based on reference to water was obtained by Formula (2):

[Math. 2]

MRI-CNR=(“Average of Signal Values within ROI of Metal Aqueous Solution Part”−“Average of Signal Values within ROI of Pure Water Part”)/“Standard Deviation of Signal Value in ROI of Background Part” (2)

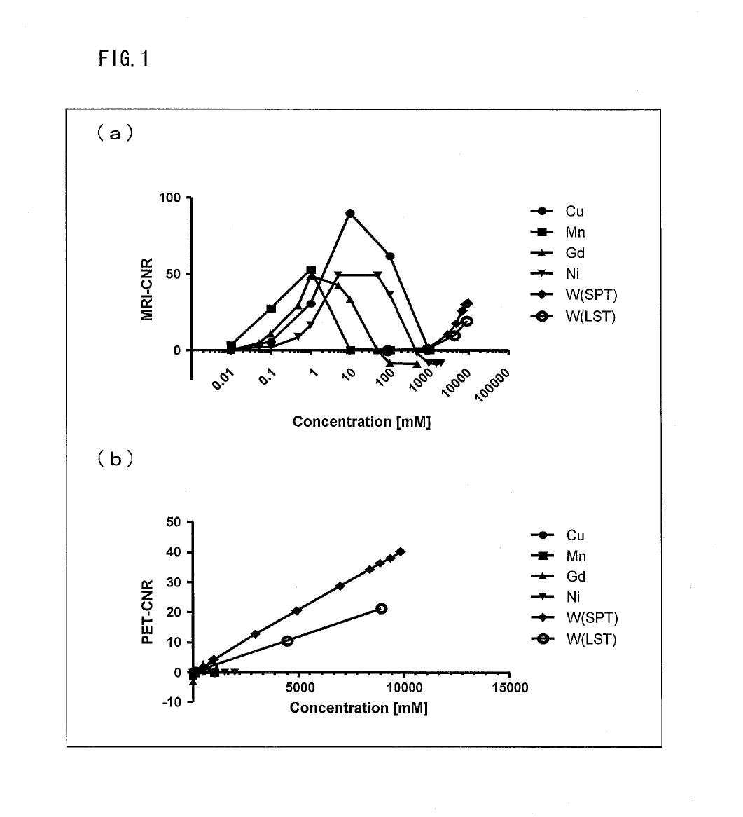

FIG. 1 shows a result showing the association between the concentrations of metals in aqueous solutions and image contrasts. In FIG. 1, metal concentrations are plotted on the horizontal axis, and MRI signal values (a) or PET absorption coefficients (b) are plotted on the vertical axis.

In the MRI images of the copper compound aqueous solutions, the manganese compound aqueous solutions, the nickel compound aqueous solutions, and the gadolinium compound aqueous solutions, increased signal values as well as increased concentrations were observed in a low-concentration range, but abrupt decreases in signal value were observed at certain concentrations (1 to 10 mM) or higher ((a) of FIG. 1). Meanwhile, in the case of the tungsten compound aqueous solutions (both the SPT aqueous solutions and the LST aqueous solutions), no signals were detected in a low-concentration range (<10 mM) in which there were increases in signal of the copper compound aqueous solutions, the manganese compound aqueous solutions, the nickel compound aqueous solutions, and the gadolinium compound aqueous solutions, but signals were detected in a high-concentration range (>100 mM) in which there were abrupt decreases in signal values of these aqueous solutions; furthermore, higher contrasts were observed at higher concentrations ((a) of FIG. 1). The degrees of contrast generated by W were substantially the same as, albeit lower than, the degrees of contrast generated by Gd, Ni, Cu, and Mn. The highest value (90) of maximum MRI contrast was exhibited by the copper compound aqueous solutions. The gadolinium compound aqueous solutions (49), the nickel compound aqueous solutions (49), and the manganese compound aqueous solutions (53) exhibited high values of maximum MRI contrast. The aqueous solutions of the tungsten compounds exhibited substantially the same and slightly lower values (LST: 20, SPT: 31). The commercially available multimodal marker, which served as a control, exhibited a comparatively low MRI contrast of 17. Table 1 shows the values (MRI-CNR_{MAX}) and concentrations at which the metal compound aqueous solutions exhibited maximum contrasts, respectively.

The PET images showed that the tungsten compound aqueous solutions generated the strongest contrast and the other aqueous solutions were about the same in absorption as water ((b) of FIG. 1). The tungsten compound aqueous solutions (both the LST aqueous solutions and the SPT aqueous solutions) exhibited substantially linear increases in contrast depending on the concentrations, i.e. exhibited higher contrasts at higher concentrations. While the aqueous solutions of manganese, nickel, copper, and gadolinium mostly exhibited very low PET contrast values (PET-CNR) of 0 to 3 at the concentrations at which they exhibited maximum contrast in MRI, the tungsten compound aqueous solutions exhibited higher PET contract values (LST: 40, SPT: 21) at higher concentrations. It should be noted that the commercially available multimodal imaging marker exhibited a very low contrast of 0.8 in PET (Table 1).

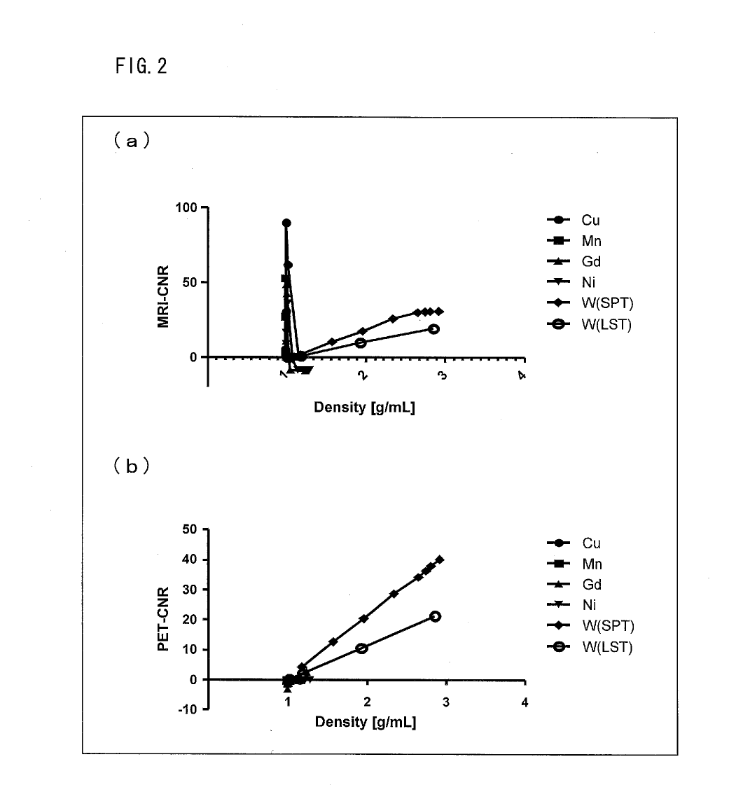

FIG. 2 shows a result showing the association between the densities of the aqueous solutions used in FIG. 1 and image contrasts. In FIG. 2, the densities of the aqueous solutions are plotted on the horizontal axis, and MRI contrasts (a) and PET contrasts (b) are plotted on the vertical axis. FIG. 2 shows that the tungsten compound aqueous solutions (both the LST aqueous solution and the SPT aqueous solutions) have very high densities (maximum densities of 2.9 to 3.0) and higher densities lead to higher contrasts in both MRI and PET.

From among these measurement results, Table 1 shows the results of measurement of the metal concentrations, densities, PET contrasts of the transition-metal compound aqueous solutions that exhibited maximum MRI contrasts.

TABLE 1
Metal concentrations, densities, PET absorption values, and
CT contrasts of transition-metal compound aqueous
solutions that exhibited maximum MRI contrasts

| | | | Metal | | CT |
|---|---|---|---|---|---|
| | MRI | Density | concentration | PET | CNR |
| Targets | CNRₘₐₓ | [g/mL] | [mM] | CNR | (×10²) |
| Transition | | | | | |
| metals | | | | | |
| (compounds) | | | | | |
| Mn (MnCl₂) | 53 | 1.0 | 1.0 | −1.4 | −0.6 |
| Ni (NiSO₄) | 49 | 1.0 | 10 | 0.57 | −0.5 |
| Cu (CuSO₄) | 90 | 1.0 | 10 | −0.32 | −0.5 |
| Gd (Gd-DTPA) | 49 | 1.0 | 1.0 | −0.66 | −0.6 |
| W (LST) | 20 | 2.9 | 8.9 × 10³ | 21.4 | 15 |
| W (SPT) | 31 | 3.0 | 10 × 10³ | 40.3 | 17 |
| Control | | | | | |
| “Multimodal | 17 | — | — | 0.8 | −0.2 |
| Marker” (IZI) | | | | | |

MRI-CNRₘₐₓ: Maximum contrast ratios of metal aqueous solutions at multiple levels of concentration,
CT-CNR: Contrast ratios of the aqueous solutions.

Furthermore, image contrasts of the representative transition-metal compound aqueous solutions shown in Table 1 were also studied by performing CT imaging on them. CT imaging was performed using an animal CT apparatus (Inveon; manufactured by Siemens AG). On PET absorption images and CT images, a region of interest (ROI) was set in the aqueous solution part in each of the tubes. For PET, the average of absorption coefficients within the ROI was obtained, and for CT, the average of CT values within the ROI was obtained. Further, an ROI was set in the background part, and a CT image contrast ratio (CT-CNR) was calculated by Formula (3):

[Math. 3]

CT-CNR=(“Average of CT Values within ROI of Metal Aqueous Solution Part”−“Average of CT Values within ROI of Pure Water Part”)/“Standard Deviation of CT Value in ROI of Background Part” (3)

As in the case of PET, only the tungsten compound aqueous solutions exhibited high CT image contrast ratios (LST: 15×10², SPT: 17×10²). On the other hand, the aqueous solutions of the gadolinium compound, the manganese compound, the nickel compound, and the copper compound only generated about the same CT values as that generated by water.

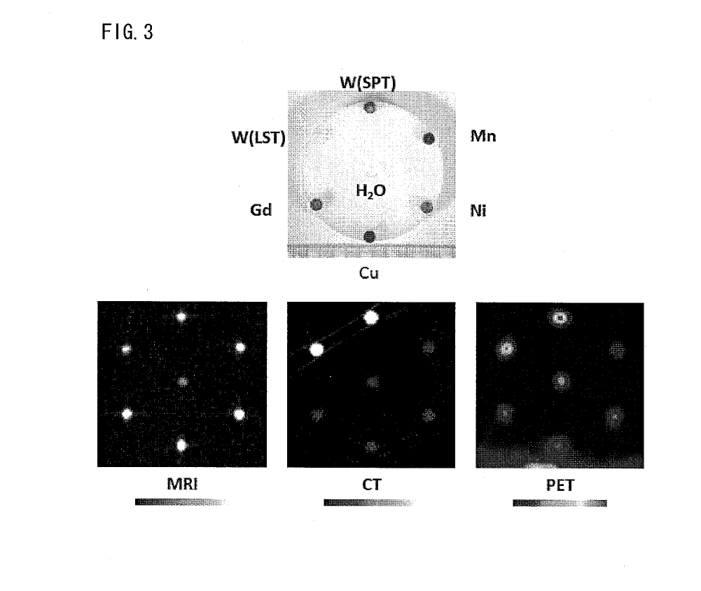

FIG. 3 shows examples of MRI, PET, and CT imaging of the representative transition-metal compound aqueous solutions named in Table 1, with the transition-metal compound aqueous solutions put in each separate minute container (that is in the shape of a cylinder having an inner diameter of 3 mm and a length of 3 mm). The imaging conditions are the same as those mentioned earlier. FIG. 3 shows that in MRI, every one of the metal compound aqueous solutions exhibits a higher contrast than water (H₂O) does and that in PET and CT, only the aqueous solutions of the tungsten (W) compounds (both LST and SPT) exhibit satisfactory contrasts.

In the case of the MRI images of the copper compound aqueous solutions, the manganese compound aqueous solutions, the nickel compound aqueous solutions, and the gadolinium compound aqueous solutions, a clear linearity between the concentrations (or densities) of the aqueous solutions and the image contrasts was observed only in a low-concentration range out of the range of concentrations at which measurements were performed. These aqueous solutions hardly generated image contrasts in PET in the range of concentrations at which measurements were performed. On the other hand, the tungsten compound aqueous solutions, which are examples of the present invention, generated increases in concentration (or density) and high contrasts in MRI, PET, and CT, and were thus found to generate satisfactory contrasts even in a range of high concentrations that are close to those of saturated solutions. It should be noted that the commercially available multimodal imaging marker did not generate a satisfactory image contrast.

Judging from these results, it can be said that as compared with the conventional MRI contrasts agents, the tungsten compound aqueous solutions of the present invention have suitable properties as materials that generate satisfactory contrasts in MRI images, CT images, and PET images.

Next, for clarification of the properties of tungsten as an MRI contrast agent, the index called “relaxivity” of each of the metals was measured and compared. Relaxivity, which indicates the performance of a contrast material for use in MRI, is expressed as an amount of change in T1 relaxivity (reciprocal of a T1 value) and T2 relaxivity (reciprocal of a T2 value) per unit concentration, and a greater value of relaxivity means that T1 and T2 contrasts are generated at a lower concentration. T1 relaxivity and T2 relaxivity were measured with aqueous solutions in which the transition metals (Mn, Ni, Cu, Gd, and W) examined above were contained in varying concentrations. By so doing, comparison and investigation were made to see how tungsten differs in relaxivity from the conventional MRI contrast materials.

Two milliliters of each of the aqueous solutions of the aforementioned metals (Mn, Cu, Ni, Gd, and W) in varying concentrations were put into each separate microtube. The microtubes were hermetically sealed. The weight of each of the aqueous solutions was measured, and the density of the aqueous solution was calculated. These microtubes were fixed on a fixed base made of styrene foam, and the T1 value and T2 value of each of the solutions were measured. For T1 value measurement, the spin echo (SE) technique was employed to acquire images with a fixed period of echo time (TE) of 7 msec and various periods of echo repetition time (TR) of 100, 200, 300, 400, 600, 800, 1000, 1500, 2000, 3000, and 4000 msec. For T2 value measurement, the spin echo technique was similarly employed to acquire images with a fixed period of TR of 3000 msec and various periods of TE of 7, 50, 100, 150, and 200 msec. In the MRI image acquisition, imaging was performed after the fixed base on which the microtubes had been fixed was placed in a region in the imaging apparatus and active shimming was performed in advance to correct magnetostatic field inhomogeneity in the region.

Imaging was performed in a constant environment at a room temperature of 22° C. A region of interest was set in an aqueous solution part of each of the tubes on reconstructed images, and the average of MRI signal values within the region of interest was obtained. An optimum T1 value was calculated by the least-squares method according to Formula (4) below with use of a data set obtained with the variations in TR.

[Math. 4]

S=M₀·(1−e^{-TR/T1}) (4)

Further, an optimum T2 value was calculated by the least-squares method according to Formula (5) below with use of a data set obtained with the variations in TE.

[Math. 5]

S=M₀·e^{-TE/T2} (5)

Next, from the T1 values and T2 values thus calculated, T1 relaxation rates (=1/T1) and T2 relaxation rates (=1/T2) were calculated, respectively. Optimum values of T1 relaxivity (r1) and T2 relaxivity (r2) were calculated by plotting these relaxation rates on the vertical axis (Unit: sec⁻¹) and the concentrations C of the metal compound aqueous solutions on the horizontal axis (Unit: mM) and fitting Formulas (6) and (7) below by the least-squares method. This relaxivity (Unit: mM⁻¹·sec⁻¹) indicates a change in relaxation rate per amount of change in unit concentration of a metal compound aqueous solution, and is widely commonly used as an index to indicate the MRI contrast ability of a contrast medium or the like.

[Math. 6]

1/T₁=1/T_{1(C=0)}+r1·C (6)

[Math. 7]

1/T₂=1/T_{2(C=0)}+r2·C (7)

Table 2 shows the T1 relaxivity and T2 relaxivity thus calculated of Gd, Ni, Cu, Mn, and W.

TABLE 2
Relaxivity of Transition-metal Compound Aqueous Solutions

| Transition metals | | |
|---|---|---|
| (compounds) | r1 [mM−1 · sec⁻¹] | r2 [mM−1 · sec⁻¹] |
| Mn (MnCl₂) | 5.5 | 120 |
| Ni (NiSO₄) | 0.61 | 0.92 |
| Cu (CuSO₄) | 0.63 | 0.65 |
| Gd (Gd-DTPA) | 4.2 | 5.6 |
| W (LST) | 0.92 × 10⁻⁴ | 0.83 × 10⁻⁴ |
| W (SPT) | 3.6 × 10⁻⁴ | 4.8 × 10⁻⁴ |

r1: T1 relaxivity,
r2: T2 relaxivity.

As has conventionally been known, Gd exhibited very high relaxivity (r1=4.2, r2=5.6 mM⁻¹·sec⁻¹), and exhibited the high ability to generate image contrasts at low concentrations. The r1 values and r2 values obtained were close to those presented in a document (Zong et al., Magn Reson Med., 53(4), p. 835-842, 2005). Mn exhibited about the same as or slightly higher relaxivity than Gd (r1=5.5, r2=5.6 mM⁻¹·sec⁻¹). Ni and Cu both exhibited high relaxivity (Ni: r1=0.61, r2=0.92, Cu: r1=0.63, r2=0.65 mM⁻¹·sec⁻¹), albeit slightly lower than that of Gd.

On the other hand, the tungsten compounds exhibited extremely weak relaxivity (SPT: r1=3.6×10⁻⁴, r2=4.8×10⁻⁴, LST: r1=0.92×10⁻⁴, r2=0.83×10⁻⁴ mM⁻¹·sec⁻¹).

These results showed that the tungsten compounds cannot generate image contrasts at lower concentrations (10 mM or lower) as do the aqueous solutions of the compounds of Mn, Ni, Cu, and Gd. On the other hand, these results showed that in a region of high concentrations of 10 mM or higher, the aqueous solutions of the compounds of Mn, Ni, Cu, and Gd produced weaker signals with extremely increased T2 shortening in addition to T1 shortening, and that, the tungsten compound aqueous solutions, to the contrary, generate image contrasts with T1 shortening finally becoming predominant in this region.

The cause of an image contrast of a transition-metal compound aqueous solution in MRI is considered to be based mainly on a magnetic property (magnetic susceptibility) depending on an electron shell state peculiar to the transition metal. However, the property varies depending on the physical state and/or the chemical state in the compound of the metal. Interaction with protons contained in water molecules present in the vicinity of the compound causes time shortening of T1 relaxation and/or T2 relaxation, and this time shortening is considered to generate an image contrast in MRI. The generation of strong signals by Ni and Gd in a low-concentration range is considered to be because both substances have highly paramagnetic metal elemental ions and therefore have high relaxivity as their properties. However, the reduction in signal value in a high-concentration range is considered to be because the extreme shortening of T2 relaxation time as well as T1 relaxation time caused weaker signals under ordinary conditions where anatomical images of living organisms are taken ((a) of FIG. 1, (a) of FIG. 2). The generation of high MRI signal values by the tungsten compound aqueous solutions in a high-concentration range and the generation of satisfactory MIR image contrasts are considered to be because the paramagnetism of tungsten ions were so weak that T1 relaxivity was very low and because T2 did not extremely lower in this concentration range.

The high-concentration tungsten compound aqueous solutions generated satisfactory image contrasts in both PET absorption images and CT images. This is considered to be because gamma rays and X-rays are easily absorbable due to the fact that the liquids are high in density, the fact that tungsten has a large atomic number, etc. On the other hand, due to limitations of solubility, it is impossible to prepare high-concentration and high-density aqueous solutions from the transition metals (Mn, Cu, Ni, and Gd) conventionally used in MRI, and this is considered to be the reason why they did not generate image contrasts in PET and CT.

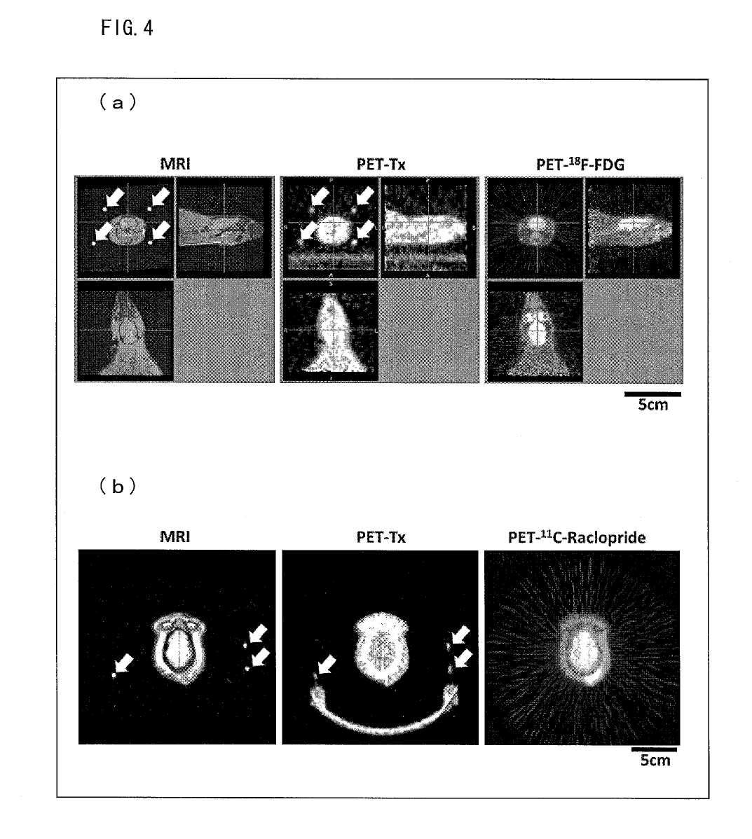

Finally, an example of an experiment in which multimodal imaging was performed on small-sized animals and medium-sized animals with multimodal markers attached is shown. Four multimodal markers of the present invention were placed at four respective positions in the area around the head of a small-sized animal (rat), and PET transmission images (PET-Tx) were taken. Further, after 30-minute PET imaging (PET-¹⁸F-FDG) following the administration of ¹⁸F-FDG, which reflects tissue glucose metabolism, MRI images were taken by the MPRAGE technique. The multimodal marker used was minute containers containing a high-concentration SPT aqueous solution. Position correction was performed on each of the images by identifying the positions of the four markers and aligning the positions of one of the images with those of another. The results are shown in (a) of FIG. 4 (in which the multimodal markers are indicated by arrows). Further, (b) of FIG. 4 shows images obtained by placing three multimodal markers at three respective positions in the area around the head of a medium-sized animal, taking PET and MRI images, and aligning the positions on one of the images with those on another. PET transmission images (PET-Tx) were taken. Then, after the administration of ¹¹C-Raclopride to the animal, 60-minute PET imaging (PET-¹¹C-Raclopride) was performed. After that, MRI images were taken by the MPRAGE technique. Position correction was performed on the PET and MRI images on the basis of marker positions (indicated by arrows).

These Examples show that since the formation of images of multimodal markers with high contrast in the same images as subjects makes it possible to easily perform registration of a plurality of images.

To sum up all of these Examples, the imaging marker of the present invention generates a clear contrast in any of images of different modalities such as MRI, PET, and CT. This makes it possible to easily perform registration by identifying the barycenter of the same marker on different images and calculating a degree of shift in position of the barycenter. This property of the maker is due to the fact that the transition metal contained therein has low relaxivity and the property of dissolving in a solution in high concentration and high density.

That is, the present invention can be in any of the following aspects:

[1] An imaging marker including a liquid containing a transition metal belonging to any of the fifth to seventh periods of the periodic table or a compound thereof.

[2] The imaging marker of 1, wherein the transition metal or the compound thereof has a concentration of 100 mM or higher in the liquid.

[3] The imaging marker of 1 to 2, wherein the imaging marker has a density of 1.2 g/mL or higher as a transition-metal compound solution.

[4] The imaging marker of 1 to 3, wherein the transition metal has a T1 relaxivity of 0.1 mM⁻¹·sec⁻¹ or lower.

[5] The imaging marker of 1 to 4, wherein the imaging marker is either an aqueous solution of sodium polytungstate or an aqueous solution of lithium polytungstate.

[6] A method of obtaining data for use in diagnostic imaging, including the steps of:

(a) imaging a subject and an imaging marker; and

(b) generating an image of the subject and an image of the imaging marker,

the imaging marker including a liquid containing a transition metal belonging to any of the fifth to seventh periods of the periodic table or a compound thereof.

[7] The method of 6, wherein during imaging, the imaging marker is attached or pasted to a body surface of the subject or to a holding member holding the subject.

[8] The method of 6 to 7, further including the step of (c), prior to step (a), attaching or pasting the imaging marker to a body surface of the subject or to a holding member that holds the subject.

[9] The method of 8, wherein all of the steps are executed without the imaging marker being removed.

[10] The method of 6 to 9, wherein during imaging, the imaging marker is introduced into a body of the subject.

[11] The method of 10, further including the step of (d), prior to step (a), introducing the imaging marker into a body of the subject.

[12] The method of 6, wherein:

step (a) is executed a plurality of times with use of a different imaging technique every time; and

step (b) is executed a plurality of times in correspondence with step (a).

[13] The method of 12, further including the step of (e) superimposing a plurality of images of the imaging marker that were generated by executing step (a) a plurality of times.

[14] The method of 12 to 13, wherein during imaging, the imaging marker is attached or pasted to a body surface of the subject or to a holding member holding the subject.

[15] The method of 14, further including the step of (c) attaching or pasting the imaging marker to a body surface of the subject or to a holding member that holds the subject.

[16] The method of 12 to 15, wherein during imaging, the imaging marker is introduced into a body of the subject.

[17] The method of 16, further including the step of (d), prior to step (a), introducing the imaging marker into a body of the subject.

[18] The method of 6 to 17, wherein the imaging marker has a concentration of 100 mM or higher of the transition metal or the compound thereof in the liquid.

[19] The method of 6 to 18, wherein the imaging marker has a density of 1.2 g/mL or higher as a transition-metal compound solution.

[20] The method of 6 to 19, wherein the transition metal has a T1 relaxivity of 0.1 mM⁻¹·sec⁻¹ or lower.

[21] The method of 6 to 20, wherein the imaging marker is either an aqueous solution of sodium polytungstate or an aqueous solution of lithium polytungstate.

[22] A diagnostic imaging system including: an imaging marker including a liquid containing a transition metal belonging to any of the fifth to seventh periods of the periodic table or a compound thereof; a holding section that holds a subject to be imaged; an imaging section that images the subject and the imaging marker; an image generation section that generates an image of the subject and an image of the imaging marker; and a display section that displays the image of the subject and the image of the imaging marker as a single image.

[23] The system of 22, wherein the imaging marker is attached or pasted to a body surface of the subject or to the holding section before the imaging section starts imaging.

[24] The system of 22 to 23, wherein the imaging marker is a second marker that is introduced into a body of the subject before the imaging section starts imaging.

[25] The system of 22, wherein: the imaging section comprises a plurality of imaging sections; the plurality of imaging sections correspond to different imaging techniques, respectively; and the display section displays, as a single image, an image of the subject and an image of the imaging marker that were formed through the same imaging technique.

[26] The system of 25, wherein the display section superimposes and displays images of the imaging marker that are contained in a plurality of single images corresponding to the different imaging techniques.

[27] The system of 22 to 26, wherein the imaging marker has a concentration of 100 mM or higher of the transition metal or the compound thereof in the liquid.

[28] The system of 22 to 27, wherein the imaging marker has a density of 1.2 g/mL or higher as a transition-metal compound solution.

[29] The system of 22 to 28, wherein the transition metal has a T1 relaxivity of 0.1 mM⁻¹·sec⁻¹ or lower.

[30] The system of 22 to 29, wherein the imaging marker is either an aqueous solution of sodium polytungstate or an aqueous solution of lithium polytungstate.

The present invention is not limited to the description of the embodiments above, but may be altered by a skilled person within the scope of the claims. An embodiment based on a proper combination of technical means disclosed in different embodiments is encompassed in the technical scope of the present invention.

### INDUSTRIAL APPLICABILITY

Use of the present invention makes it possible to accurately and easily perform registration of images taken through different imaging techniques, so that the accurate understanding of biological phenomena and the accurate diagnosis of various diseases can be achieved through multimodal imaging. As such, the present invention can be used for the development of multimodal imaging apparatuses.

1. An imaging marker comprising
a liquid containing a transition metal belonging to any of the fifth to seventh periods of the periodic table (excluding gadolinium) or a compound thereof.

2. The imaging marker as set forth in claim 1, wherein the transition metal or the compound thereof has a concentration of 100 mM or higher in the liquid.

3. The imaging marker as set forth in claim 1, wherein the imaging marker has a density of 1.2 g/mL or higher as a transition-metal compound solution.

4. A method of obtaining data for use in diagnostic imaging, comprising the steps of:
(a) imaging a subject and an imaging marker with use of an imaging technique; and
(b) generating an image of the subject and an image of the imaging marker,
the imaging marker including a liquid containing a transition metal belonging to any of the fifth to seventh periods of the periodic table (excluding gadolinium) or a compound thereof.

5. The method as set forth in claim 4, further comprising the step of (c), prior to step (a), attaching or pasting the imaging marker to a body surface of the subject or to a holding member that holds the subject.

6. The method as set forth in claim 5, wherein all of the steps are executed without the imaging marker being removed.

7. The method as set forth in claim 4, further comprising the step of (d), prior to step (a), introducing the imaging marker into a body of the subject.

8. The method as set forth in claim 4, wherein:
step (a) is executed a plurality of times with use of a different imaging technique every time; and
step (b) is executed a plurality of times in correspondence with step (a).

9. The method as set forth in claim 8, further comprising the step of (e) superimposing a plurality of images of the imaging marker that were generated by executing step (a) a plurality of times.

10. A diagnostic imaging system comprising:
an imaging marker including a liquid containing a transition metal belonging to any of the fifth to seventh periods of the periodic table (excluding gadolinium) or a compound thereof;
a holding section that holds a subject to be imaged;
an imaging section that images the subject and the imaging marker;
an image generation section that generates an image of the subject and an image of the imaging marker; and
a display section that displays the image of the subject and the image of the imaging marker as a single image.

11. The system as set forth in claim 10, wherein the imaging marker is attached or pasted to a body surface of the subject or to the holding section before the imaging section starts imaging.

12. The system as set forth in claim 10, wherein the imaging marker is a second marker that is introduced into a body of the subject before the imaging section starts imaging.

13. The system as set forth in claim 10, wherein:
the imaging section comprises a plurality of imaging sections;
the plurality of imaging sections correspond to different imaging techniques, respectively; and
the display section displays, as a single image, an image of the subject and an image of the imaging marker that were formed through the same imaging technique.

14. The system as set forth in claim 13, wherein the display section superimposes and displays images of the imaging marker that are contained in a plurality of single images corresponding to the different imaging techniques.
